(19) United States
(12) Patent Application Publication
(10) Pub. No.: US 2005/0175556 A1
(43) Pub. Date: Aug. 11, 2005

(54) Skin Darkening (Sunless Tanning) Compositions Based on Enhancement of Melanin Synthesis by Tyrosinase Promoters

(75) Inventor: Shyam K Gupta, Scottsdale, AZ (US)

Correspondence Address: SHYAM K. GUPTA;BIODERM RESEARCH, 5221 E. WINDROSE DRIVE, SCOTTSDALE, AZ, 85254, US

(73) Assignee: BIODERM RESEARCH, Scottsdale, AZ (US)

(21) Appl. No.: 10/708,093

(22) Filed: Feb. 7, 2004

(51) Int. Cl. A61K 7/42; A61K 38/44

(52) U.S. Cl. 424/59; 424/94.4

(57) ABSTRACT

This invention discloses skin darkening (sunless tanning) compositions based on enhancement of melanin synthesis by tyrosinase enzyme by providing essential substrates for tyrosinase as well as the essential elements to activate the active-site of tyrosinase enzyme. These compositions thus also act as promoters of tyrosinase enzyme.

### BACKGROUND OF INVENTION

The cosmetic treatment of skin to produce a visible eventone has been practiced since ancient times. The use of plant-derived extracts and salves to darken or tan light colored skin has become very popular among Caucasian and light skin colored non-Caucasian cultures. Colorants or tints containing substantive dyes as their coloring component are normally used for temporary colors. Substantive dyes are based on dye molecules which are directly absorbed onto the hair or skin and do not require an oxidative process for developing the color. Dyes such as these include, for example, henna which has been used since ancient times for coloring the body and hair. Also, increased awareness of the harmful effects of tanning by means of radiation, along with a continued desire by many to be tanned, has led to an increased interest in tanning by means of chemical agents. The chemicals which are currently used in cosmetically tanning human skin include:
(i) Agents that react with skin proteins and nucleotides to form a color complex, such as dihydroxyacetone, erythrulose, glyceraldehyde, 6-aldo-D-fructose, hydroxymethyl-glyoxal, mucondialdehyde, malealdehyde, substituted succindialdehydes, methylglyoxal (pyruvaldehyde) and substituted 4,4′-dihydroxypyrazolin-5-ones.
(ii) Bronzing agents such as juglone (for example, U.S. Pat. No. 6,395,261, Laforet), lawsone (for example, U.S. Pat. Nos. 5,569,460; 3,920,808; and 4,708,865), and henna. Some of these may also be reactive with skin proteins and nucleotides.
(iii) Skin surface coloring agents such as various synthetic dyes, natural colorants (such as caramel and carmine), and iron oxides (for example, U.S. Pat. No. 6,033,648; and U.S. Patent Application 20040018161).
(iv) The combinations of the foregoing tanning agents.

The color of human skin is differentiated by the nature and quantity of natural pigment, melanin, present in the epidermal layers of skin. The formation of melanin from amino acid tyrosine involves several biogenetic steps mediated by enzyme tyrosinase. Tyrosine is first oxidized by tyrosinase to dihydroxy phenylalanine (l-dopa), then to dopaquinone. Dopaquinone is then converted into eumelanin (black, white, and Asian skin types; skin color dependent on the quantity of eumelanin in skin), or phaeomelanin (red-haired skin types). This conversion proceeds through the intermediate formation of leucodopachrome and dopachrome (eumelanin), or cysteinyldopa (phaeomelanin). Tyrosine, L-DOPA, glutathione, and copper thus play a major role in the formation of melanin via the action of tyrosinase enzyme on its various substrates.

The enhancement of skin darkening effect for cosmetic appearance has been practiced by several procedures in the prior art. Various colorants, both synthetic and from natural sources, have been applied to skin. These generally wash off easily and can cause staining of fabric and clothing. Skin reactive compositions, such as dihydroxyacetone (DHA), erythrulose, lawsone, juglone, glyceraldehyde, 6-aldo-D-fructose, hydroxymethylglyoxal, mucondialdehyde, malealdehyde, substituted succindialdehydes, methylglyoxal (pyruvaldehyde), substituted 4,4′-dihydroxypyrazolin-5-ones and certain anthraquinones (for example, U.S. Pat. No. 6,660,284; Bordier et al.) and their silicone derivatives (U.S. Patent Application 20030185769, Ehlis et al.) have been utilized with some success. These compositions, which contain a reactive ketone or aldehyde chemical group, generally react with the amino group of certain amino acids present in skin protein to form Schiff's bases. Such Schiff's bases are usually colored compositions that impart coloration to the topical layers of skin. However, it should be pointed out that neither the artificial coloring of skin with colorants nor the application of skin reactive compositions (even the combinations of such skin reactive compositions, such as those disclosed in U.S. Patent Applications 20030044365, Candau; 20020031482, Schreier; 20010043909, SaNogueira et al.; U.S. Pat. Nos. 6,113,888, Castro et al., 6,231,837, Stroud et al., 6,214,322, Castro et al.; 5,569,460, Kurz et al.) to produce topical skin coloration results in the natural development of skin color by the formation of melanin. For this reason, most of the skin colors produced by the above procedures do not correspond exactly to skin coloration of the consumer using such compositions. It would thus be highly desirable to activate body's own mechanism of color formation (melanin synthesis), which would then result in the darkening of skin in the same natural shade of melanin of that consumer. Also, in certain cosmetic applications, consumer may desire temporary darkening of skin by skin colorants or skin reactive compositions in combination with more permanent natural darkening of skin.

It is the purpose of this invention to disclose compositions that activate body's own mechanism of skin darkening effect. This is achieved by the enhancement of the activity of tyrosinase enzyme with concurrent supply of the key substrates required for melanin synthesis by tyrosinase enzyme. Such comprehensive, natural mode of skin darkening effect is unprecedented in the prior art, and an unexpected discovery by the present inventor. Moreover, the compositions of the present invention are compatible in combination with compositions for temporary coloring or tanning of skin with colorants or skin reactive compositions. This provides both a short-term and a more natural longer-lasting skin darkening effect.

U.S. Patent Application 20040005288 (Lin et al.) discloses skin darkening compositions based on He Shou Wu extract in combination with a pigment, such as melanin. However, He Shou Wu extract is not known to be a part of biochemical mechanisms that involve body's own synthesis of melanin via tyrosinase enzyme. In addition, topical coloring agents are also utilized in combination with He Shou Wu, which can result in unnatural skin darkening effect and unwanted staining of clothing.

U.S. Patent Application 20020155138 (Martin et al.) discloses Hedychium extract for regulating skin tone and coloration. Any specific examples of skin darkening compositions based on this extract were not disclosed by these inventors.

The use of UV lamps for skin darkening is also commonly practiced. UV exposure, however, results in accelerated skin aging and increased incidence of skin cancer. The ability to generate a tanned appearance without incurring photo-damage is important to consumers. This seriously limits the utility of UV lamps for safe skin darkening applications.

U.S. Patent Application 20020192738 (Brissette et al.) discloses a tyrosinase assay that can be used for in-vitro testing of tyrosinase activator compositions.

U.S. Patent Application 20040013617 (Rick et al.), U.S. Pat. Nos. 6,645,474 (Galdi et al.), 6,451,293 (Schreier et al.), 6,261,541 (Karpov et al.), 6,231,837 (Stroud et al.), 5,972,314 (Crotty et al.), 5,942,212 (Lentini et al.), 5,612,044 (Suares et al.), and 6,482,397 (Scott et al.) disclose sunless tanning composition based on DHA (either alone, or in combination with erythrulose). DHA, currently the most widely used of the self-tanning agents, is believed to exert its effect through interactions between its ketone group and the amino groups of amino acids and peptides naturally occurring in the hydrolipid pellicle and first layers of the stratum corneum of the skin. Such Maillard reactions are believed to lead to formation of brown pigments in the skin, thereby giving it an appearance similar to that of a naturally obtained tan. Although DHA-based, and other, self tanning agent-containing compositions are currently in widely accepted use, they do suffer from several attendant limitations, chief amongst which are a “streaky” tan, primarily resulting from the uneven application of the compositions to the skin of end users. Such unevenness primarily arises from the difficulty the users have in seeing the sunless tanning compositions once they have applied them to their skin, and hence, in ensuring that the compositions have been evenly applied. Moreover, these compositions do not accelerate body's own synthesis of melanin by tyrosinase activation.

U.S. Pat. No. 6,656,455 (Laughlin) discloses a spray methodology to cover skin surface with a coloring composition for skin darkening. Artificial tanning has been known for more than 40 years, with artificial tanning products appearing on the U.S. market as early as 1959. The two key types of tanning processes are by colorants and bronzers. Tanning by colorants is based on the color reaction which occurs between components of the skin and the colorant. The most commonly used chemical for artificial tanning is dihydroxyacetone (DHA). DHA reacts solely with the stratum corneum. It interacts with amines, peptides and free amino acids to generate a Maillard reaction. The resulting products are cyclic and linear polymers that have a yellow or brown color. Two common bronzers are juglone and lawsone. Both are naphthoquinones. When applied to skin, lawsone produces an orange hue and juglone produces a greenish-brown tan. They are sometimes used in combination with DHA to modify the color or hue of the tan or to intensify the color. As should be clear, this methodology does not provide any activation of tyrosinase enzyme for melanin synthesis.

U.S. Patent Application 20030228268 (Candau) discloses a skin reactive composition, similar to DHA (in that it being a ketone derivative, 1,7-bisphenyl heptane-3,5-dione) that does not provide any tyrosinase enzyme activation benefits for natural skin color pigmentation. U.S. Patent Application 20040009200 (Seyler) discloses other compositions based on 1,7-diphenyl-3,5-heptanedione for skin pigmentation.

U.S. Patent Application 20020166182 (Bhagyalakshmi et al.) discloses compositions based on a botanical coloring agent from Mucuna which, upon oxidation with an oxidizing composition, imparts coloration to human hair or skin. Such coloration of skin is external and not caused by the activation of tyrosinase enzyme. Moreover, such colorations of skin do not impart darkening effects natural to particular consumer″s skin. Also, this system for coloring hair and/or skin comprising at least three separately packaged components: (a) a thio compound capable of cutting the cysteine bond and an alkaline reagent which may or may not be separately packaged (b) Mucuna and (c) an oxidizing agent. The coloring system may be suitably supplied in the form of a combination kit. A method of dyeing skin and/or hair is also provided comprising the sequential steps of application of the thio compound and alkaline reagent followed by the application of Mucuna and finally applying the oxidizing agent. Such a treatment scheme is very complex, expensive and inconvenient to consumer.

U.S. Pat. No. 6,399,046 (Schonrock et al.) discloses certain polyphenolic compounds, for example, extracts from leaves of plants of the Theales order with the Theaceae family, in particular the species Camellia spec., very particularly the tea types Camellia sinensis, C. assamica, C. taliensis and C. irrawadiensis increase the activity of melanocytes in human skin and intensify natural skin tanning. However, it is well known that such polyphenolic compounds also act as strong antioxidants, and antioxidants are known to inhibit the activity of tyrosinase enzyme.

U.S. Pat. No. 5,866,133 (Kim et al.) discloses Caesapinia sappan extract promotes the activity of tyrosinase, which is the most important enzyme involved in melanin synthesis in melanocytes. Caesapinia sappan is a plant belonging to the bean family. Caesapinia sappan L. has been used as a red dye. The coloring components of Caesapinia sappan, brazilin and hematoxylin, have been used as a hair dye. It is not thus clear if the skin darkening effect of this extract is exclusively due to the activation of tyrosinase, or from a combination of effects that includes skin coloration by brazilin and hematoxylin present in such extracts.

U.S. Pat. No. 6,033,648 (Candau) discloses compositions based on particulate pigments, such as iron oxides, for temporary body bronzing effects. Such body decorative methods do not affect the body's own production of melanin by tyrosinase activation.

U.S. Pat. No. 5,989,876 (Belcour-Castro et al.) discloses compositions based on colorants obtained from certain plants, which upon chemical modification with a quinone are useful for coloring hair and skin.

U.S. Pat. Nos. 5,643,554 (Menon et al.), 5,961,991 (Wenke et al.), and 4,968,497 (Wolfram et al.), disclose synthetic derivatives of melanin for sunscreen, hair dye, and body coloration applications.

U.S. Pat. No. 4,609,544 (Herlihy) discloses compositions based on certain dye precursors, which upon oxidation with a chemical oxidizing agent provide compositions useful for artificial tanning of skin. Such compositions do not provide any tyrosinase activation effects, and the colorations are not natural to body's own melanin color.

U.S. Patent Application 20030040496 (Chandler et al.) and U.S. Pat. Nos. 6,639,121 (DePinho et al.) and 6,037,329 (Baird et al.) disclose the role of tyrosinase promoters in the treatment of cancer. Such tyrosinase promoters can theoretically be of use in skin darkening compositions, although their reduction to such practice may be very difficult or prohibitively expensive.

U.S. Patent Application 20030232743 (Seiberg et al.) discloses certain peptides for skin darkening applications. U.S. Patent Application 200200347729 (Orlow et al.) discloses certain P-proteins for the management of skin pigmentation.

From the above prior art knowledge it is clear that skin darkening (sunless tanning) compositions based on the principle of the enhancement of body's own synthesis of melanin are practically non-existent. Since melanin is produced by the action of enzyme tyrosinase on its various substrates (such as tyrosine or dihydroxyphenyl alanine), it would be highly desirable to develop compositions that are based on the enhancement of tyrosinase enzyme for the production of melanin. The color of melanin thus produced would match more closely to the natural color of melanin of the consumer using such compositions.

Accordingly, the present invention discloses compositions that are based the enhancement of tyrosinase enzyme by providing essential substrates for tyrosinase as well as the essential elements to activate the active-site of tyrosinase enzyme. This scheme thus provides a complete system for tyrosinase cascade. This is further discussed in the Detailed Description section of this invention.

### SUMMARY OF INVENTION

This invention discloses skin darkening (sunless tanning) compositions based on enhancement of melanin synthesis by tyrosinase enzyme by providing essential substrates for tyrosinase as well as the essential elements to activate the active-site of tyrosinase enzyme. These compositions thus also act as promoters of tyrosinase enzyme.

### DETAILED DESCRIPTION

Tyrosine, L-DOPA, glutathione, and copper play a major role in the formation of melanin via the action of tyrosinase enzyme. Tyrosinase is a copper-based oxido-reductase enzyme. The active-site of tyrosinase is highly accessible to external ligands. Since tyrosinase is dependent on copper, the external supply of copper can be used for the activation of this enzyme. However, it is well known that copper in unbound state can be very toxic to living cells, as discussed in U.S. patent application Ser. No. 10/306,948 (Gupta). Copper in complexed state is usually not bioavailable. It has also become known that ATP, a major nucleotide present in human body, plays a major role in copper transport, in the form of copper transporting ATPase enzyme that utilizes the energy of ATP-hydrolysis to transport copper from the cytosol through various cell membranes.

The following key moieties are required for proper functioning of tyrosinase in a living cell for optimal production of melanin; (i) Ample supply of tyrosinase substrate (for example, tyrosine or dihydroxy phenylalanine) for melanin synthesis; and (ii) An activator composition for the activation of the active-site of tyrosinase enzyme.

The activation of the active-site of tyrosinase enzyme is dependent on the following: (i) A bioavailable source of copper (Tyrosinase is a copper-based oxido-reductase enzyme); (ii) A transporter(s) of copper from extra-cellular to intra-cellular levels; (iii) A storage device for copper in a bound state within the cell; and (iv) An energy source for the transport of copper from copper storage molecule to the apoprotein of tyrosinase.

Accordingly, the present invention is based on the enhancement of tyrosinase enzyme by providing essential substrates for tyrosinase as well as the essential elements to activate the active-site of tyrosinase enzyme. This is achieved by providing the following compositions: (i) Ample supply of tyrosinase substrate (for example, tyrosine or dihydroxy phenylalanine) for melanin synthesis; (ii) A bioavailable source of copper; (iii) A transporter(s) of copper from extra-cellular to intra-cellular levels; (iv) A storage device for copper in a bound state within the cell; and (v) An energy source for the transport of copper from copper storage molecule to the apoprotein of tyrosinase.

These are further discussed below.
(i) Tyrosinase Substrates: Tyrosinase enzyme can use either tyrosine or dihydroxy phenylalanine as a substrate for the synthesis of dopaquinone, which is a precursor of melanin in the biochemical pathway of tyrosinase action. Tyrosine, as a substrate, is first converted into dihydroxy phenylalanine by tyrosinase. Dihydroxy phenylalanine is then converted into dopaquinone by the same enzyme. It would thus be advantageous to provide dihydroxy phenylalanine or its bioavailable chemical transformants as a substrate, as it would already be next stage substrate, thus not requiring an additional oxidation step by tyrosinase (which would be the case if tyrosine is used as a substrate).
(ii) Bioavailable Source of Copper: Tyrosinase requires copper in its active-site. Without copper (II), tyrosinase can not function. It is of further importance, since various forms of copper can have significantly different biological or cosmetic functions. Copper biomolecules can occur in four types of copper centers. These four copper types, and their characterization methodologies, are noted below.

(a) Copper (I), (b) Normal Copper (II), (c) Blue Copper (II), and (d) Coupled (Cu^{II})₂. While many copper biomolecules contain copper in only one form, for example “blue” or “normal”, there are also numerous cases where several different types of copper are present and that can provide difficulties in working out their mode of action, or even their applications. The “normal” copper (II) sites are those in which Cu²⁺ ion is coordinated by a square set of ligands, usually all nitrogen atoms, such as those present in imidazole moiety of one (or several) histidine molecules. There may be additional ligands occupying more distant coordination sites above and below that square plane of nitrogen ligands.

The “blue” copper (II) state entails environment quite unlike those in “normal” copper (II) tetragonal complexes. Numerous sophisticated spectroscopic analyses have been made of both the biomolecules themselves and their model systems. It is to be noted that copper in “blue” copper (II) sites is electronically bound to four different atoms, two of which are nitrogen and two of which are sulfur atoms.

Coupled (Cu^{II})₂ is found most commonly in respiratory proteins of phyla Mollusca and Anthropoda, for example squid, octopus, lobster, and crabs. These proteins, called hemocyanins, are very large that contains subunits. Each subunit contains a pair of Cu atoms, and those atoms can bind one molecule of oxygen per pair of copper atoms. The two-copper active site of hemocyanins is also found in enzyme tyrosinase. In humans this enzyme converts phenols to catechols that leads to the eventual formation of skin pigment, melanin. It is to be noted that copper in “coupled” (Cu^{II})₂ is electronically bound to a minimum of four different atoms, two of which are nitrogen and two of which can be oxygen. Copper must thus be provided in a bound, bioavailable form via both oxygen and nitrogen electron-donor binding centers. Also, copper, in the form of its various inorganic salts (such as copper sulfate, copper chloride, etc.) or chelated salt forms (such as copper acetate, copper gluconate, copper EDTA complex, copper amino acid complex, etc.) has poor absorption through topical layers of skin and not available to tyrosinase. This is because copper is easily bound by amino acids, such as lysine, arginine, and histidine present in topical layers of skin protein. Free copper ions are also very toxic to living cells. It has now been discovered that copper bound to certain organic acids or chelating agents that are stronger in their copper binding strength than the above mentioned amino acids on skin surface is more bioavailable for tyrosinase. Examples of such bioavailable forms of copper include, but not limited to Copper ATP, Copper cystine complex, Copper cysteine complex, Copper N-acetyl-cystine complex, Copper N-acetyl-cysteine complex, Copper tyrosine complex, Copper L-DOPA complex, Copper Mucuna prurience complex, Copper glutathione complex, Copper carnosine complex, and such. Such complexes of copper can either be made in situ by the combination of appropriate copper derivatives and binding agent or used in a pre-manufactured state. An example of the in-situ preparation of copper ATP is by the reaction of copper gluconate with disodium ATP in a water solution, which results in the formation of copper ATP and sodium gluconate in-situ. Such in-situ preparation of tyrosinase desirable copper derivatives is thus very convenient and inexpensive.
(iii) Transporter of Copper: The bioavailable forms of copper mentioned in Section (i) above also act as transporters of copper from the topical layers of skin into deeper layers of skin. ATP, glutathione, and N-acetyl-cysteine are especially useful transporter of copper.
(iv) Intracellular Copper Storage: Both ATP and glutathione are very useful for the intracellular storage of copper in a non-toxic form. It is well known that living cell prefers to store copper as copper glutathione complex. An external supply of glutathione is thus highly beneficial.
(v) Energy Source for Intracellular Copper Transport: ATP and fructose phosphates are well known for this function. An external supply of ATP or fructose phosphate is thus highly beneficial.

It is thus clear to those versed in the art that by providing copper in a bound state as copper ATP or copper glutathione several benefits are achieved. Copper is first transported (as copper ATP, for example) from the topical layers of skin into living cells of skin where tyrosinase enzyme resides. Copper is provided there in a bound state (as copper glutathione, for example). A source of energy (as ATP) is also provided for the transportation of copper from its storage site to the active-site of enzyme tyrosinase. The constituents required for the activation of tyrosinase cascade are thus fully provided by the teachings of the present invention.

### EXAMPLES

The following examples are presented to illustrate presently preferred practice thereof. As illustrations they are not intended to limit the scope of the invention. All quantities are in weight %.

### Example 1

Skin Darkening Serum. Ingredients (1) Deionized water 49.3 (2) Mucuna prurience extract 15.0 (3) Methylpropanediol 30.0 (4) Dimethicone copolyol 4.0 (5) Glutathione (reduced) 0.5 (6) Copper Adenosine Triphosphate (Cu ATP) 0.2 (7) Preservatives 0.5 (8) Carbomer 0.5. Procedure. Make main batch by mixing 1, 2, 5, 6, and 7 at room temperature. Pre-mix 3 and 4 to a solution and add to main batch with mixing. Adjust pH to 4.0 6.5 range.

### Example 2

Skin Darkening Cream. Ingredients (1) Deionized water 73.0 (2) Cetearyl alcohol (and) Dicetyl phosphate (and) Ceteth-10 phosphate 5.0 (3) Cetyl alcohol 2.0 (4) Glyceryl stearate (and) PEG-100 stearate 4.0 (5) Caprylic/capric triglyceride 5.0 (6) Mucuna prurience extract 10.0 (7) Copper Glutathione 0.5 (8) Preservatives 0.5. Procedure. Mix 1 to 5 and heat to 75-80° C. Adjust pH to 4.0-4.5. Cool to 35-40° C. with mixing. Add 6 to 8 with mixing. Adjust pH to 4.0-6.5. Off-white cream.

### Example 3

Skin Darkening Cream in Combination with Henna Colorant Combination. Ingredients (1) Deionized water 68.0 (2) Cetearyl alcohol (and) Dicetyl phosphate (and) Ceteth-10 phosphate 5.0 (3) Cetyl alcohol 2.0 (4) Glyceryl stearate (and) PEG-100 stearate 4.0 (5) Caprylic/capric triglyceride 5.0 (6) Mucuna prurience extract 10.0 (7) Copper Glutathione 0.5 (8) Preservatives 0.5. (9) Henna extract 5.0. Procedure. Mix 1 to 5 and heat to 75-80° C. Adjust pH to 4.0-4.5. Cool to 35-40° C. with mixing. Add 6 to 9 with mixing. Adjust pH to 4.0-6.5, if necessary. Off-white cream.

### Example 4

Skin Darkening Cream in Combination with DHA Skin Reactive Colorant Combination. Ingredients (1) Deionized water 68.0 (2) Cetearyl alcohol (and) Dicetyl phosphate (and) Ceteth-10 phosphate 5.0 (3) Cetyl alcohol 2.0 (4) Glyceryl stearate (and) PEG-100 stearate 4.0 (5) Caprylic/capric triglyceride 5.0 (6) Mucuna prurience extract 10.0 (7) Copper Glutathione 0.5 (8) Preservatives 0.5. (9) Dihydroxyacetone (DHA) 5.0. Procedure. Mix 1 to 5 and heat to 75-80° C. Adjust pH to 3.5-4.5. Cool to 35-40° C. with mixing. Add 6 to 9 with mixing. Adjust pH to 3.5-6.0. Off-white cream.

### Example 5

Sprayable Skin Darkening Lotion. Ingredients (1) Deionized water 47.62 (2) Lauramidoyl Inulin 0.6 (3) Xanthan Gum 0.18 (4) Distarch Phosphate 4.0 (5) Hydrogenated Polydecene 3.0 (6) Isostearyl Isostearate 8.5 (7) PEG-8 Dimethicone 3.0 (8) Cyclomethicone and PPG-15 Stearyl Ether 12.5 (9) Sorbitan Isostearate 1.0 (10) Hydroxyethylacrylate Sodium and Acryloyl Dimethyltaurate Copolymer and Squalane and Polysorbate-60 1.5 (11) Tocopheryl Acetate 0.5 (12) Phenoxyethanol and Parabens 0.5 (13) Dihydroxy phenylalanine 10.0 (14) Copper Glutathione and Copper ATP and Copper Tyrosine complex 1.5 (15) Aloe Barbadensis 5.0 (16) Fragrance 0.6. Procedure. Disperse 2 in 1 and add 3 and 4 with mixing. Heat to 40-45° C. Add 5 to 11 with stirring for 15 minutes. Add 12 with mixing. Add 13 and 14 to main batch with mixing. Cool to 30-35° C., add 15 and 16, then cool to room temperature with mixing. Adjust pH to 4.5 6.5 range.

### Example 6

Skin Darkening Clear Gel with Synthetic Dyes Combination. Ingredients (1) Deionized water 68.6 (2) Preservative 0.7 (3) Ammonium Acryloyldimethyltaurate/VP Copolymer 2.0 (4) Glycerin 15.0 (5) Copper Glutathionate 0.5 (6) Copper Carnosine 0.2 (7) Polysorbate-20 2.0 (8) Dihydroxy Phenylalanine 10.0 (9) Basic Brown #99 0.55 (10) Basic Red #76 0.15 (11) Basic Yellow #57 0.3. Procedure. Mix (1) to (4) till a clear gel is formed. Heat at 40 to 50 C and add all other ingredients with mixing. Cool to room temperature. Adjust pH to 3.5 4.0 range.

1. A topical skin darkening or skin tanning composition comprising:
(i) At least one tyrosinase substrate composition for melanin synthesis boost, and
(ii) At least one tyrosinase activator composition, and
(iii) A cosmetically or pharmaceutically suitable carrier composition or delivery system.

2. A topical skin darkening or skin tanning composition comprising:
(i) At least one tyrosinase substrate composition for melanin synthesis boost, and
(ii) At least one tyrosinase activator composition, and
(iii) At least one skin surface coloring composition, and
(iv) A cosmetically or pharmaceutically suitable carrier composition or delivery system.

3. A composition according to claim 1 wherein,
(i) From about 0.0001% to about 60% of at least one tyrosinase substrate composition for melanin synthesis boost, and
(ii) From about 0.0001% to about 20% of at least one tyrosinase activator composition, and,
(iii) From about 1% to about 99% of a cosmetically or pharmaceutically suitable carrier composition or delivery system.

4. A composition according to claim 1 wherein tyrosinase substrate composition can be selected from, but not limited to tyrosine, dihydroxy phenylalanine, L-DOPA, Mucuna prurience extract, catechol, and their chemical derivatives.

5. A composition according to claim 1 wherein tyrosinase activator composition can be selected from, but not limited to copper ATP, Glutathione, Cystine, Cysteine, N-Acetyl-Cystine, N-Acetyl-Cysteine, copper cystine complex, copper cysteine complex, copper N-acetyl-cystine complex, copper N-acetyl-cysteine complex, copper tyrosine complex, copper L-DOPA complex, copper Mucuna prurience complex, copper glutathione complex, copper carnosine complex, various copper chelates in which copper is concurrently bound with both oxygen and nitrogen electron donors, and combinations thereof.

6. A composition according to claim 1 wherein cosmetically or pharmaceutically acceptable delivery system or carrier base can optionally include additional skin beneficial ingredients selected from skin cleansers, surfactants (cationic, anionic, non-ionic, amphoteric, and zwitterionic), skin and hair conditioning agents, vitamins, hormones, minerals, plant extracts, anti-inflammatory agents, concentrates of plant extracts, emollients, moisturizers, skin protectants, humectants, silicones, skin soothing ingredients, analgesics, skin penetration enhancers, solubilizers, moisturizers, emollients, anesthetics, colorants, perfumes, preservatives, seeds, broken seed nut shells, silica, clays, beads, luffa particles, polyethylene balls, mica, pH adjusters, processing aids, and combinations thereof.

7. A composition according to claim 1 wherein a cosmetically acceptable delivery system or a carrier base can be selected in the form of a lotion, cream, gel, spray, thin liquid, body splash, mask, serum, solid cosmetic stick, lip balm, shampoo, liquid soap, bar soap, bath oil, cologne, hair conditioner, salve, collodion, impregnated patch, impregnated strip, skin surface implant, and any other such cosmetically or pharmaceutically acceptable topical delivery forms.

8. The compositions according to claim 1 wherein the cosmetically or pharmaceutically acceptable delivery system can be traditional water and oil emulsions, suspensions, colloids, microemulsions, clear solutions, suspensions of nanoparticles, emulsions of nanoparticles, powders, or anhydrous compositions.

9. A composition according to claim 2 wherein,
(i) From about 0.0001% to about 60% of at least one tyrosinase substrate composition for melanin synthesis boost, and
(ii) From about 0.0001% to about 20% of at least one tyrosinase activator composition and,
(iii) From about 0.0001% to about 40% of at least one skin surface coloring composition, and
(iv) From about 1% to about 99% of a cosmetically or pharmaceutically suitable carrier composition or delivery system.

10. A composition according to claim 2, wherein skin surface coloring composition can be selected from, but not limited to dihydroxyacetone (DHA), lawsone, juglone, erythrulose, glyceraldehyde, 6-aldo-D-fructose, hydroxymethylglyoxal, mucondialdehyde, malealdehyde, substituted succindialdehydes, methylglyoxal (pyruvaldehyde) and substituted 4,4′-dihydroxypyrazolin-5-ones, henna, various Arianor dyes, melanin, caramel, charcoal, and combinations thereof.

11. A composition according to claim 2 wherein cosmetically or pharmaceutically acceptable delivery system or carrier base can optionally include additional skin beneficial ingredients selected from skin cleansers, surfactants (cationic, anionic, non-ionic, amphoteric, and zwitterionic), skin and hair conditioning agents, vitamins, hormones, minerals, plant extracts, anti-inflammatory agents, concentrates of plant extracts, emollients, moisturizers, skin protectants, humectants, silicones, skin soothing ingredients, analgesics, skin penetration enhancers, solubilizers, moisturizers, emollients, anesthetics, colorants, perfumes, preservatives, seeds, broken seed nut shells, silica, clays, beads, luffa particles, polyethylene balls, mica, pH adjusters, processing aids, and combinations thereof.

12. A composition according to claim 2 wherein a cosmetically acceptable delivery system or a carrier base can be selected in the form of a lotion, cream, gel, spray, thin liquid, body splash, mask, serum, solid cosmetic stick, lip balm, shampoo, liquid soap, bar soap, bath oil, cologne, hair conditioner, salve, collodion, impregnated patch, impregnated strip, skin surface implant, and any other such cosmetically or pharmaceutically acceptable topical delivery forms.

13. The compositions according to claim 2 wherein the cosmetically or pharmaceutically acceptable delivery system can be traditional water and oil emulsions, suspensions, colloids, microemulsions, clear solutions, suspensions of nanoparticles, emulsions of nanoparticles, powders, or anhydrous compositions.
